(19) United States
(12) Patent Application Publication
(10) Pub. No.: US 2026/0115294 A1
(43) Pub. Date: Apr. 30, 2026

(54) DERIVATIVES OF 5-HYDROXYLATED POLYMETHOXYFLAVONES WITH ENHANCED BIOAVAILABILITY FOR ORAL DELIVERY AND COMPOSITIONS THEREOF

(71) Applicant: DEEPSENSE BIOTECHNOLOGIES INC., WESTPORT, CT (US)

(72) Inventors: Johan Brag, Westport, CT (US); Shiming Li, Glastonbury, CT (US)

(21) Appl. No.: 18/930,942

(22) Filed: Oct. 29, 2024

(51) Int. Cl. A61K 47/54

(52) U.S. Cl. CPC A61K 47/543; A61K 47/544

(57) ABSTRACT

We are disclosing derivatives of 5-hydroxylated polymethoxyflavones. The derivatives offer enhanced oral bioavailability and improved intestinal permeability. The derivatives of the invention release the biologically active 5-hydroxylated polymethoxyflavones that are activated by chemical or enzymatic processes in the GI Tract. The derivatives are constructed as bioreversible prodrugs containing the bioactive molecules covalently linked with lipids like fatty acids, glycerides or phospholipids. The derivatives are useful against inflammatory conditions and aging. Formulations including the derivatives are also disclosed.

### TECHNICAL FIELD

The present invention relates to derivatives of polymethoxylated flavones. More particularly, the present invention relates to derivatives of 5-hydroxylated polymethoxyflavones with enhanced bioavailability for oral delivery. These compounds are useful in the treatment of a variety of metabolic disorders and inflammatory diseases.

### BACKGROUND

Polymethoxylated flavones (PMFs) have been shown to exhibit a broad spectrum of biological activity, including anti-inflammatory, anti-carcinogenic, anti-tumor, anti-viral, anti-thrombogenic and anti-atherogenic properties. PMFs exist almost exclusively in the citrus genus, especially in peels of mandarins (Citrus reticulate). PMFs are poorly soluble in water and only moderately soluble on lipids, limiting their oral bioavailability. The preferred choice for encapsulation of poorly soluble drugs, the addition of extra surfactants, solubilizers, or large amounts of amphiphilic materials in drug formulations can inevitably increase the risk of hyposensitivity and immunogenicity.

5-Hydroxylated PMFs (5HPMFs) are natural metabolites of PMFs and have also been isolated from aged citrus peels. These 5-PMFs are characterized by the replacement of a methoxy group by a hydroxyl group at the C-5 position on the A ring. Several 5HPMFs have been identified in aged citrus peels including 5-hydroxy-6,7,8,3′,4′-pentamethoxyflavone (5-demethynobiletin), 5-hydroxy-6,7,8,4′-tetramethoxyflavone (5-demethyltangeretin), and 5-hydroxy-3,6,7,8,3′,4′-hexamethoxyflavone among others.

Previous structure-activity relationship studies suggested that demethylation-modified analogues of the methoxy group at the C-5 position on the A ring of PMFs have more profound bioactivities including inhibiting cancer and inflammation than the parent PMFs. This may be due to the existence of lone pairs of electrons on the oxygen atoms of neighboring methoxy and carbonyl groups forming a stable six-membered ring.

In a study by Wang et al. (2008), 5-demethyl nobiletin and 5-demethyl tangeretin were found to have a stronger ability to reduce the lipid peroxidation damage than their corresponding parent counterparts nobiletin and tangeretin. A study by Li et al. (2007) compared the growth inhibitory activity of six PMFs and nine DPMFs by screening against human HL-60 cancer cell lines. The 5-DPMFs were found to have greater inhibitory activity than their methylated counterparts.

PMFs and their hydroxylated PMF derivatives have poor solubility in pharmaceutically acceptable solvents which has prevented their further clinical development. In addition to the hydrophobic nature of multiple methoxy groups, PMFs are aglycones (forms lacking sugar moieties), which leads to low solubility in water. This poor water solubility may cause slower dissolution, which typically limits absorption after oral intake.

Because of these problems, the use of PMFs and their 5-hydroxylated derivatives in pharmaceutical and nutraceutical compositions have proved problematic owing to the limited bioavailability of these compounds. Accordingly, there has continued to exist a need to be effective and safe formulations of PMFs and 5HPMFs with high bioavailability for oral delivery.

Various strategies have been proposed to overcome the limited bioavailability of flavonoids including emulsions, nano-encapsulation and prodrugs. Wang et al. has proposed encapsulating PMFs in citrus oil emulsion-based delivery systems for oral delivery of PMFs as nutraceuticals in food and beverages. Generally, such approaches are not satisfactory as the drug loading in such emulsions is limited to around 1% in carrier oils with additional excipients, surfactants and stabilizers substantially reducing the ultimate potency and stability of the compounds.

There is therefore a need for stable and concentrated PMF compositions with high solubility for improved absorption in the GI tract.

### SUMMARY OF THE INVENTION

The invention relates to novel polymethoxylated flavone (PMF) derivatives with improved oral bioavailability. PMFs have very poor aqueous solubility and low lipid solubility. Accordingly, it is an object of the present disclosure to provide PMF derivatives with enhanced absorption, distribution, metabolism, and excretion (ADME) properties.

This disclosure specifically teaches prodrugs of 5-hydroxy polymethoxylated flavones (5HPMFs) with improved bioavailability. The prodrugs of the invention release the biologically active molecules (5HPMFs) that are activated by chemical or enzymatic processes in the GI Tract to release the active metabolite. They contain the bioactive molecules covalently linked with lipids like fatty acids, glycerides or phospholipids.

It is an object of the invention to provide novel derivatives of 5-hydroxy polymethoxylated flavones (5HPMFs) with improved oral bioavailability.

It is a further object of the disclosure to provide novel derivatives of 5-H-PMFs engineered to overcome low absorption in the GI Tract.

It is another object of the invention to reduce enzymatic degradation in the intestines.

It is still another object of the invention to target the lymphatic route.

It is further object of the derivatives to improve the pharmacokinetics of 5HPMFs.

In a preferred embodiment, the 5HPMF is is selected from 5-hydroxy-6,7,8,3′,4′-pentamethoxyflavone (5-demethyl-nobiletin), 5-hydroxy-6,7,8,4′-tetramethoxyflavone (5-demethyl-tangeretin), 5-hydroxy-6,7,3′,4′-tetramethoxyflavone (5-demethyl-sinensetin), and 5-hydroxy-3,6,7,8,3′,4′-hexamethoxy-flavone.

It is yet another object to provide compositions including the prodrugs and mixtures thereof suitable for use in nutraceutical and nutritional supplement formulations.

This disclosure teaches compounds of the structural formula I below wherein R5 is a pharmaceutically acceptable moiety selected to improve the oral bioavailability of the hydroxylated polymethoxylated flavone and wherein the 5HPMF comprises at least 4 methoxy groups and wherein R3, R3′ R4′ and R5′ are OCH3, OH or H, where R6, R7 are OCH3 and where R8 is OCH3 or H.

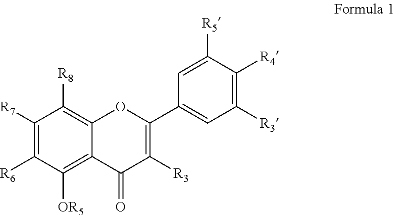

In a preferred embodiment, R5′ is H and R8 is OCH₃.

In another preferred embodiment, R5 is a phosphoryl functional group.

The compounds of the disclosure are novel derivatives of 5-H-PMFs which the inventors have found to have enhanced bioavailability and which are especially suitable for use in compositions formulated for oral delivery.

### DESCRIPTION OF FIGURES

FIG. 1 is an illustrates the polymethoxyflavones of the disclosure.

FIG. 2 illustrates hydroxylated polymethoxyflavones of the disclosure.

FIG. 3 illustrates the chemical formula of the claimed compounds of the disclosure

FIG. 4 illustrates an embodiment of a fatty acid ester of 5 demethyl nobiletin.

FIG. 5 illustrates embodiments of phospholipid prodrugs of the disclosure

FIG. 6 illustrates a triglyceride prodrug of the disclosure

FIG. 7 illustrates phosphate esters of the disclosure.

FIG. 8. Illustrates glycoside esters of the disclosure

FIG. 9 illustrates an embodiment of PEGilated prodrugs of 5-demethyl nobiletin.

FIG. 10 illustrates an embodiment of a carbmate prodrug of 5 demethyl nobiletin.

FIG. 11 illustrates an embodiment of the synthesis of a 5-hydroxy-polymethoxyflavone.

FIG. 12 illustrates the synthesis of 5-demethyl-sinensetin

FIG. 13 illustrates methods for synthesizing 5-acyl ester prodrugs of the disclosure.

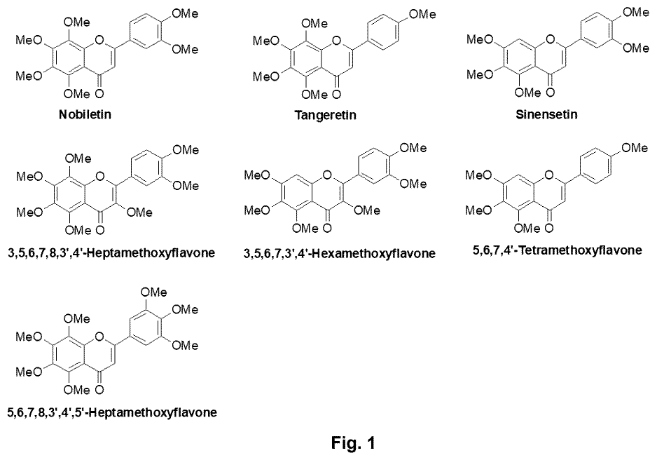

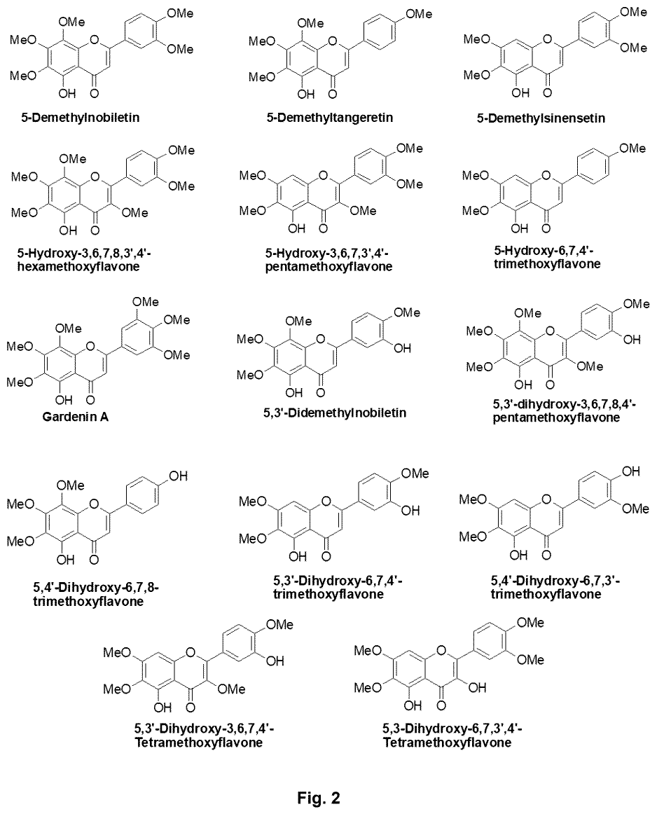

### DETAILED DESCRIPTION

Flavonoids represent the largest group of compounds among phytochemicals. Flavonoids are secondary metabolites in plants that are part of the polyphenol category. They are made up of two aromatic carbon rings that are connected by a three-carbon bridge. More than 8000 compounds with flavonoid structure have been identified. Flavonoids have been found to have many health benefits, including a lower risk of heart disease and all-cause mortality. However, in general flavonoids have low bioavailability due to limited absorption, extensive metabolism and rapid excretion.

Most flavonoids are often poorly lipid soluble. The lipophilicity of flavonoids and their ability to interact with the cell membrane are important factors that influence their pharmacological activity. Many flavonoids present a number of hydroxyl groups that provide some polarity and weak acidic properties to the molecules. The inverse correlation between the number of hydroxyl groups and the lipophilicity of flavonoids has been demonstrated experimentally. The bioactivities of flavonoids also depend on their chemical structure, which may show substitutions such as hydrogenations, methylations, malonylations, sulphatations, and glycosylations.

Polymethoxylated flavones, exclusively found in the peels of certain citrus varieties, have been found to have greater metabolic stability bioavailability and pharmacologic activity than polyhydroxylated flavonoids and therefore offer a much greater potential as drug leads. Nevertheless, PMFs have certain limitations including poor aqueous solubility which reduces their oral bioavailability. Their hydrophobic nature is partly derived from the multiple methoxy groups. Nobiletin in particular, has a poor water solubility making oral delivery difficult. One alternative to improving bioavailability of PMFs has been the development of prodrug derivatives for oral delivery.

The most common PMFs in citrus peels are tangeretin (5,6,7,8,4′-pentamethoxyflavone), nobiletin (5,6,7,8,3′,4′-hexamethoxyflavone), 3,5,6,7,8,3′,4′-heptamethoxyflavone, sinensetin (5,6,7,3′,4′-pentamethoxyflavone), and 3,5,6,7,3′,4′-hexamethoxyflavone, which structurally contain five to seven methoxy groups in different positions.

The inventors have discovered that 5-hydroxylated PMFs (5HPMFs) exhibit improved stability and efficacy over the non-hydroxylated PMFs in the treatment of various disorders including metabolic, mood, inflammatory and cognitive disorders. Like other PMFs, these hydroxylated derivatives have suboptimal pharmacokinetic properties including poor solubility and membrane permeability limiting their use as lead compounds for various therapies.

In various embodiments of the disclosure the 5HPMFs is selected from 5-hydroxy-6,7,8,3′,4′-pentamethoxyflavone (5-demethynobiletin), 5-hydroxy-6,7,8,4′-tetramethoxyflavone (5-demethyltangeretin), 5-hydroxy3,6,7,8,3′,4′ hexamethoxyflavone, 5-hydroxy-6,7,3′4′-tetramethoxyflavone (anisomalin), 5,3′-dihydroxy-6,7,4′-trimethoxyflavone (Eupatorin), 5,4′-dihydroxy-6,7,3′-trimethoxyflavone (Cirsilineol), 5,3′-dihydroxy-3,6,7,8,4′-pentamethoxyflavone, 5,2′-dihydroxy-6,7,8,6′-tetramethoxyflavone (Skullcapflavone II), 5-hydroxy-6,7,4′-trimethoxyflavone (Salvigenin), 3,5-dihydroxy-6,7,8-trimethoxyflavone and 5-hydroxy-6,7,8,3′,4′,5′-hexamethoxyflavone (Gardenin A). (See table 1 below)

TABLE 1

| PMF | R3 | R5 | R6 | R7 | R8 | R3′ | R4′ | R5′ |
|---|---|---|---|---|---|---|---|---|
| Gardenin A | H | OH | OCH3 | OCH3 | OCH3 | OCH3 | OCH3 | OCH3 |
| 5-hydroxy 3,6,7,8,3′4′ | OCH3 | OH | OCH3 | OCH3 | OCH3 | OCH3 | OCH3 | H |
| hexamethoxyflavone | | | | | | | | |
| 5-demethyl nobiletin | H | OH | OCH3 | OCH3 | OCH3 | OCH3 | OCH3 | H |
| 5,3′-dihydroxy 3,6,7,8, | OCH3 | OH | OCH3 | OCH3 | OCH3 | OH | OCH3 | H |
| pentamethoxyflavone | | | | | | | | |
| 5-demethyl tangeretin | H | OH | OCH3 | OCH3 | OCH3 | H | OCH3 | H |
| 5-demethyl sinensetin | H | OH | OCH3 | OCH3 | H | OCH3 | OCH3 | H |
| Gardenin D | H | OH | OCH3 | OCH3 | OCH3 | OH | OCH3 | H |
| Eupatorin | H | OH | OCH3 | OCH3 | H | OH | OCH3 | H |
| Anisomalin | H | OH | H | OCH3 | OCH3 | H | H | H |
| Cirsineol | H | OH | OCH3 | OCH3 | H | H | OH | H |
| Salvigenin | H | OH | OCH3 | OCH3 | H | H | OH | H |

In a preferred embodiment, the 5HPMFs is selected from 5-hydroxy-6,7,8,3′,4′-pentamethoxyflavone (5-demethynobiletin), 5-hydroxy-6,7,8,4′-tetramethoxyflavone (5-demethyltangeretin) and 5-hydroxy3,6,7,8,3′,4′ hexamethoxyflavone.

Studies have shown compounds need to meet at least four conditions effective passive delivery through the small intestine: 1) molecular weight of less than around 500 Dalton, 2) sufficient aqueous and lipid solubility with a Log P (octanol/water) of between 2 and 4, 3) potent drugs requiring low concentrations for therapeutic effect. See Table 2 below.

TABLE 2

| | MW | | | MW |
|---|---|---|---|---|
| PMF | (Da) | LogP | HYDROXYLATED PMF | (Da) |
| Tangeretin | 372 | 1.76 | 5-demethyltangeretin | 358 |
| Nobiletin | 402 | 2.02 | 5-demethylnobiletin | 388 |
| HeptaMF | 432 | 2.42 | 5-demethylheptaMF | 418 |

Small molecular weight is a major feature of most flavonoids and PMFs in particular. The hydroxylated PMFs all have molecular weights of less than 500 Dalton and preferred PMF prodrugs of the disclosure are also small. See Table 1 above.

PMFs are generally more lipophilic than non-methoxylated flavones because of their methyl groups and absence of hydroxyl groups. This makes them less suited for oral delivery. This requires incorporating the PMFs in suitable lipophilic solvents such as fatty acid esters. Various additives and surfactants may also be used to enhance permeation.

In a first embodiment, it is an object of the disclosure to increase bioavailability by covalently modifying the 5-H-PMF by attaching a lipophilic moiety (fatty acid, phospholipid or glyceride) to increase lipid solubility and small intestine permeability.

In another embodiment, it is an object of the disclosure to increase bioavailability by covalently modifying the 5HPMF by attaching hydrophilic moiety (e.g., phosphate, sulfate, glucose, PEG) to increase the water solubility of the 5HPMF.

### Derivatives of 5-Hydroxylated Polymethoxyflavones

An object of the current invention is to is to modify the physicochemical properties of the 5HPMFs in order to improve its bioavailability for oral drug delivery.

The derivatives of the disclosure are engineered as prodrugs. The prodrugs are pharmacologically inactive compounds that are activated in the body after their administration.

The prodrugs of the disclosure are bioreversible derivatives of 5HPMFs. The inventors have engineered improved derivatives of 5HPMFs, specifically prodrug derivatives with significantly improved pharmacokinetics over the parent compound. The prodrugs are used to optimize the bioavailability or ADME (absorption, distribution, metabolism, excretion) and pharmacological properties of the 5HPMFs.

The prodrug approach of the invention facilitates the formulation and administration, drug efficacy, improves site specificity, and decreases toxicity.

The prodrug design of the invention encompasses two different approaches: Hydrophilic prodrugs (where the prodrug is more hydrophilic than the parent drug) and lipophilic prodrugs (where the prodrug is more lipophilic than the parent drug).

The disclosure teaches novel, custom engineered prodrug compounds of 5HPMFs derivatives with enhanced metabolic stability, low toxicity, improved solubility and high intestinal permeability able to reach systemic circulation where the active ingredients (5HPMFs) are released and subsequently delivered to the target tissue. By combining the 5HPMFs with various customized moieties, the resulting prodrugs can be imparted with novel physicochemical properties that contribute to better bioavailability, resulting in greater ability to reach the target tissue and greater potency.

The prodrugs are designed to improve the bioavailability of 5HPMFs and hence their therapeutic efficacy. Regeneration of the parent drug occurs in vivo by either enzymatic or chemical processes.

Since the GI tract shows a high enzymatic activity, mainly due to esterase activity, the prodrugs have been designed to offer enhanced resistance to hydrolysis and reduce first-pass metabolism during digestion.

### Lipidic Prodrugs

The intestinal capillary pathway is the most common way to absorb oral drugs, but for compounds with poor solubility and permeability and high first-pass metabolism, this pathway is very inefficient. Intestinal lymphatic transport of lipophilic drugs or prodrugs is a promising strategy to improve the oral delivery efficiency of these compounds. Lipid drug conjugates (LDCs) are chemical entities which are commonly referred to as lipidic prodrugs. The current disclosure teaches LDCs where the bioactive molecule is a hydroxylated PMF covalently linked with a lipid moiety such as a fatty acid, a triglyceride or phosphoglyceride.

An object of the LDCs of the disclosure is to increase active ingredient payload. Conjugating lipidic moieties to hydroxylated PMFs improves the fat solubility of the LDC. Essentially, LDCs allow for the hydroxylated PMFs to be absorbed in a similar fashion to other dietary fat using lymphatic transport.

Another object of the LDCs of the disclosure is to increase delivery through the lymphatic system targeting thereby improving systemic bioavailability. Highly lipophilic LDCs may be conveyed into the intestinal lymphatic system just as fat. It is estimated that log P value above 5, and solubility in TG of >50 mg/g are prerequisites for intestinal lymphatic transport. Such LDCs, can be absorbed from the lamina propria into the porous mesenteric lymphatic vessel capillary lacteal, rather than blood capillaries; the porous structure of the lacteal and the absence of basal membrane allows permeation of large colloids (200-800 mm), whereas blood capillaries have tight junctions and continuous basal membrane that limits the permeation of large colloids. This allows highly lipophilic LDCs to bypass first-pass hepatic metabolism and makes them orally bioavailable, allowing the possibility of altering the drug delivery rate to the blood and controlled drug delivery.

In various embodiments, the disclosure teaches LDCs comprising a 5-H-PMF covalently bound to a fatty acid, glycerol or phospholipid functional group.

### Fatty Acid Prodrugs

Fatty acids are most commonly used as candidates for conjugating bioactive(s) to lipids to endow them with lipophilicity. Fatty acids consist of a long acyl chain and a reactive —COOH functional group, which tend to interact with a hydroxyl functional group available with the bioactive molecule resulting in an ester linkage of high stability.

The LDCs of the disclosure are less active than the parent hydroxylated PMFs. They hydrolyze LDCs are also typically less cytotoxic than the parent bioactive compound.

In an embodiment, an LDC is formed by conjugating a 5HPMF with an unsaturated

In a preferred embodiment, the LCFA is selected from oleic acid (C18:1), linoleic acid (C18:2) or linolenic acid (C18:3) LCFAs form micellar structures leading to the formation of chylomicrons which facilitate the absorption through biological membranes

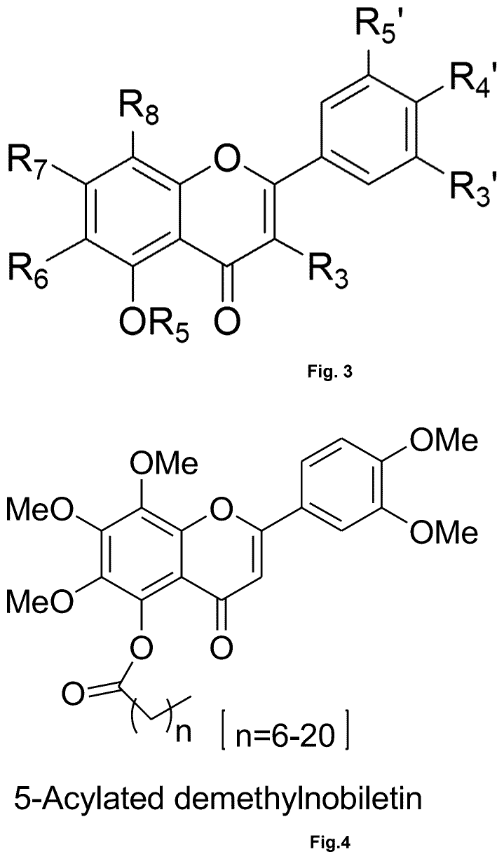

FIG. 3 illustrates the structure of a fatty acid ester prodrug of a 5-H-PMF according to the disclosure. The inventors have found that novel esters resulting from the acylation of 5-H-PMFs with (C8:C20) saturated or unsaturated fatty acids improves bioavailability for oral delivery through the small intestine.

TABLE 3
below illustrates the change in LogP between the parent compound
5 demethyl nobiletin and the same drug conjugated with lauric acid.
Attached the lauric acid moiety to the HPMF increases the LogP from
2.4 to 6.5 The longer the alkyl chain the higher the LogP.

| Compound | MW | Log P |
|---|---|---|
| 5 DMN | 388 | 2.4 |
| 5 LDN | 568 | 6.5 |

LCFAs form micellar structures leading to the formation of chylomicrons which facilitate the absorption through biological membranes

In particular, medium and long chain acids will help form micelles which can be absorbed through the lymphatic system. The prodrug will then hydrolyze in serum as the lymphatic system drains into the systemic circulation.

FIG. 4 illustrates an embodiment of an LDCs of the disclosure as a conjugate of 5-demethylnobiletin and a C6-20 fatty acid.

In another embodiment, saturated, mono-unsaturated or polyunsaturated long chain C14:C20 fatty acids including myristic acid, palmitic, myristoleic, palmitoleic, oleic, linoleic and linolenic acids are used.

### Phospholipid Prodrugs

In another embodiment, the LDC is formed by conjugating a 5HPMF with a phospholipid (PL). PLs are structurally related to fats, as both are derived from phosphatidic acid, which has the basic structure of glycerol with two ester bonds with fatty acids and one ester bond with phosphoric acid.

The structure and design of the PL LDC can highly influence their fate within the body. Careful design can direct the complex to the desired processing pathway, aiming to achieve different purposes, e.g., lymphatic transport, controlled release, and local effect at the site of inflammation.

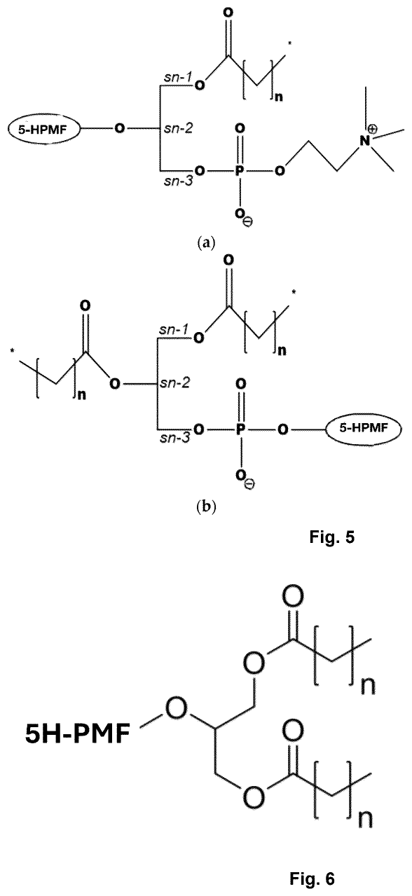

FIG. 5 illustrates various configurations of phospholipid LDCs of 5HPMF. In a first embodiment, illustrated in configuration a) the 5HPMF is conjugated at position sn-2 of the glycerol backbone. Conjugation of the 5HPMF to the sn-2 position of the PL results in PL-based conjugate that has similar surface properties and aggregation performance like natural PL.

In a preferred embodiment illustrated in configuration b) the 5HPMF is conjugated to the phosphate group. Attaching the 5HPMF to the phosphate group (5HPMF-PL) is used with the aim of avoiding the LDC hydrolysis by the PLA enzyme and hence allowing the LDC to be absorbed and incorporated into the metabolic lipid processing pathway. 5HPMF-PL can undergo lymphatic transport hereby bypassing extensive first-pass hepatic metabolism. This allows prodrugs to avoid presystemic metabolism and reach higher systemic bioavailability.

### Triglyceride Prodrugs

It is an object of the invention to disclose novel LDCs of 5HPMF that are absorbed through the same mechanism as dietary fats.

In FIG. 6 we illustrate an embodiment disclosing LDCs of 5HPMFs in the form of triglyceride mimetic prodrugs, (5HPMF-TG). The 5HPMF is typically conjugated with a diglyceride at the position sn-2. The sn-2 monoglyceride, specifically, remains intact in the intestines prior to absorption and is therefore the preferred site for HPMF attachment

In an embodiment, the diglyceride is a diglycerol ester of long chain fatty acids. In a preferred embodiment, one or more of the fatty acids of the triglyceride is lauric, palmitic, oleic, linoleic or linolenic acid.

Since the intestinal lymph is responsible for the transport of dietary lipids (primarily triglyceride, TG), this means that triglyceride-mimetic LDCs (TG-LDC) of 5HPMF can not only improve lipid solubility and permeability but also simulate the absorption process of dietary triglyceride to promote the entry of LDC molecules into the mesenteric lymphatic system. [00082]5HPMF-TG LDCs (rather than 5HPMF-FA-LDCs) offer an alternate platform to promote intestinal lymphatic transport. The LDCs are dissembled in the gastrointestinal tract to form mixed micelles that are then reassembled into chylomicrons within enterocytes and secreted into the lymph Chylomicron formation may increase the bioavailability of lipophilic compounds such as 5HPMF in a number of ways: (i) increasing the amount that is transported across the cells; (ii) protecting them from metabolism within the cells; (iii) preventing first-pass metabolism in the liver.

### Hydrophilic Prodrugs

The disclosure teaches novel prodrug designs which significantly hydrophilicity of 5-hydroxylated PMFs (5-OH-PMF) by attaching a hydrophilic moiety to increase the aqueous solubility of the prodrug. In a preferred embodiment, the hydrophilic moiety (e.g., phosphate, sulfate, glucose, PEG) to increase the aqueous solubility.

Like most flavonoids, PMFs and their 5 hydroxylated PMFs are generally categorized as BCS class IV drugs, low solubility and low permeability. Although many techniques such as solid dispersion self-emulsifying emulsion, nanoparticles have been used with PMFs in attempts to improve the oral absorption, a definitive improvement has not been achieved yet. Thus, a brand-new approach is needed.

The inventors are the first to disclose derivatives of 5HPMFs with enhanced solubility and oral bioavailability.

### Phosphate Prodrugs

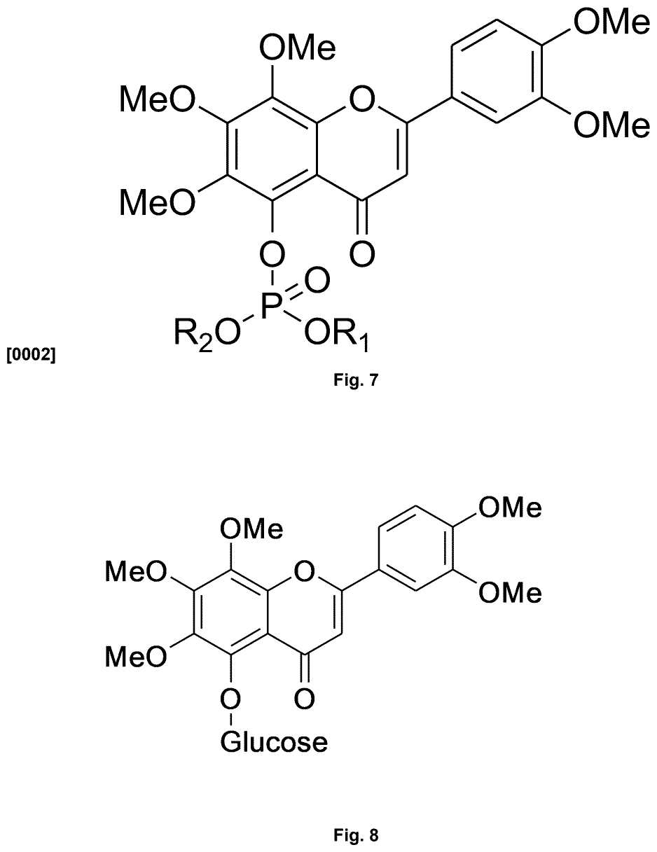

The disclosure teaches phosphate prodrugs from the ester bonding of phosphate moiety with a 5-OH-PMF. A phosphate mono- or diester is highly hydrophilic and relatively stable when free in metabolic surroundings because of its negative charge at physiological pH, but is readily activated upon complexation to various counterions in an enzyme's active site. Thus, phosphorus compounds provide optimal properties to enable them to transit biological membranes but then able to release the parent drug once inside the target cell. FIG. 7 illustrates phosphate prodrugs of 5-demethyl nobiletin where R1, R2, are H, alkyl or aryl groups.

Generally, alkyl groups provide greater resistance to hydrolysis than aryl groups. In a preferred embodiment, the organic groups attached to the phosphorus atom is an alkyl group.

Efficient delivery of a 5-OH-PMF phosphate monoester into the cell can afford important metabolic advantages. For example, nucleoside analogues undergo activation after cell entry by conversion to the corresponding mono-, di- and ultimately triphosphates, and use of a protected phosphate may allow intersection with natural metabolic processes at a later stage. This strategy may be particularly important with nucleotide prodrugs where it can allow the agent to bypass the rate-limiting initial phosphorylation. Furthermore, phosphate prodrugs may confer stability to serum phosphatases, and therefore support more effective dosing.

### Glycoside Prodrugs

The disclosure teaches hydrophilic prodrugs from the bonding of 5-hydroxylated PMFs with sugar moieties, such as glucose, galactose, or glucuronic acid, to improve the aqueous solubility of 5-OH-PMFs. In various embodiments, the sugar moiety is attached to the hydroxyl group of the 5-OH-PMF via various spacers, and these prodrugs are bioconverted to their parent drug molecules by β-glucosidase, β-galactosidase, or β-glucuronidase. However, the sugar-spacer moiety can also be hydrolyzed from the ester, carbamate, or carbonate bond between the parent drug and the spacer by esterases. FIG. 8. illustrates a 5-demethyl glucose prodrug.

### Polyethylene Glycol Prodrugs

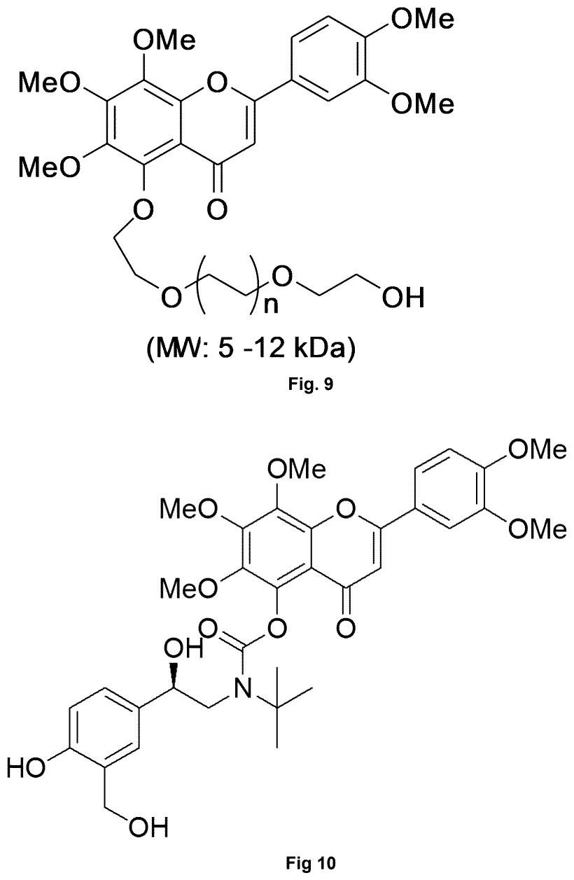

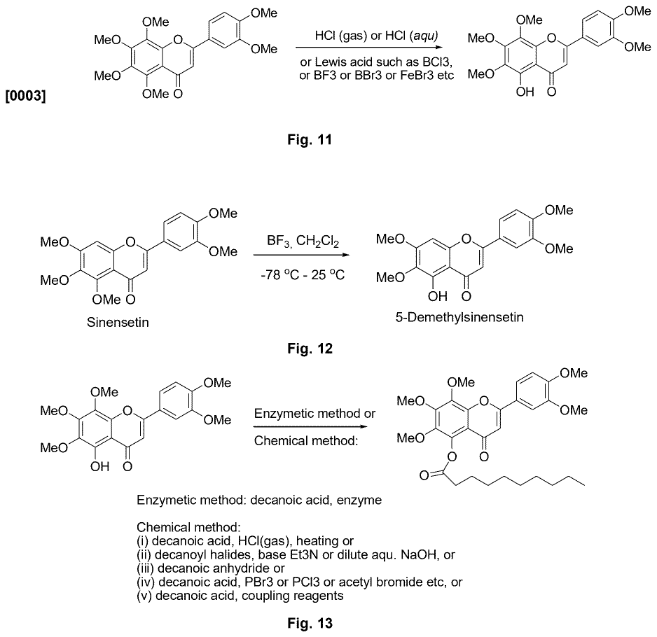

Promoieties with no ionizable or charge containing groups can also be used to increase aqueous solubility. One strategy for high-melting compounds is to decrease the melting point by masking groups that are able to form intermolecular hydrogen bonds. Examples of this approach are the polyethylene glycol prodrugs. Polyethylene glycols (PEGs) are linear or branched neutral oligomers or polymers of ethylene oxide with a broad molecular weight range (300-10,000,000 g/mol). FIG. 9 illustrates a pegylated prodrug of 5 demethyl nobiletin. These amphiphilic polymers have also been used to improve the aqueous solubility of poorly water-soluble drugs for both oral and parenteral drug delivery. PEG promoieties can be attached to the hydroxyl or amine group of the drug via various spacers and linked to the spacer via an ester, amide, carbonate, or carbamate bond that undergoes chemical cleavage.

### Carbamate Prodrugs

FIG. 10 illustrates a carbamate ester of 5-demethyl nobiletin. Carbamates are derivatives of carbamic acid, whose amino and carboxyl termini are substituted by a variety of structurally diverse alkyl, aryl, or alkyl-aryl substituents and are identified by the presence of the —O—CO—NH— linkage. Carbamate-bearing molecules play an important role in medicinal chemistry due to their chemical stability and capability to permeate cell membranes.

Carbamates have been manipulated for use in the design of prodrugs as a means of achieving first-pass and systemic hydrolytic stability. In recent years, carbamate derivatives have received much attention due to their application in drug design and discovery. To the inventors knowledge, no carbamate derivatives of hydroxylated PMFs have been developed.

In the present disclosure we teach carbamates of hydroxylated PMFs with increased resistance to hydrolysis. Without being bound by theory, carbamates are more stable than the corresponding carboxylic acid esters due to the fact that the carbonyl group is less electrophilic than the carboxyl group. The synthesis of carbamate derivatives of hydroxylated PMFs leads a wide range of novel prodrugs with increased hydrolytic stability and bioavailability.

### Lipid Formulations

In lipid formulations of the present invention, the 5HPMF derivatives are highly stable and no stabilizers, anti-oxidants or emulsifiers are needed. Moreover, preferred lipid formulations have been found to be absorbed through the lymphatic system and bypassing the GI tract metabolism.

In an embodiment of the disclosure, the formulation comprises one or more 5HPMF derivatives and a pharmaceutically acceptable solvent.

In further embodiments, the formulation includes one or more bioavailability enhancers.

In various embodiments, the formulations comprise between 1% and 30% of the 5HPMF derivative by weight and preferably between 5% and 20% by weight.

Typically, the potency of the formulation will depend on the concentration level of the PMF derivatives.

In preferred embodiments, the solvent has an HLB value of less than 5.

### Solvents

The solvent is a pharmaceutically acceptable lipid carrier.

In an embodiment the solvent is a triglyceride of a C1 2:C20 fatty acid. In preferred embodiments, the solvent is an ester of an alcohol and a fatty acid. In preferred embodiments, the alcohol is a naturally occurring alcohol. In more preferred embodiments, the alcohol is a fatty alcohol, glycerol or glycol. Examples of fatty alcohols include cetyl alcohol, stearyl, cetearyl, phenethyl pentylene glycol and propanediol.

In a preferred embodiment, the solvent is an ester of glycerol, glycol or fatty alcohol C8:C12 medium chain fatty acid.

In a preferred embodiment, the lipid solvent is glycerol monocaprylate.

### Permeation Enhancers

Examples of suitable permeation enhancers include monoesters of propylene glycol, fatty alcohols In another embodiment, the permeation enhancer is an alcohol or glycerol ester of fatty acids.

Preferred permeation enhancers include glyceryl monocaprylate/caprate and glyceryl stearate, oleyl alcohol and diethylene glycol monethyl ether (DGEE).

### EXAMPLES

### Example 1:Self-Emulsifying Lipid Based Formulation

Amounts are % by weight). Excipients were obtained from Gattefosse

| | Function | Amount |
|---|---|---|
| 5-Lauroyl demethyl nobiletin (5LDMN) | Active Ingredient | 10% |
| Glycerol Monocaprylate (GM) | Solvent | 60% |
| Propylene Glycol (PG) | Solvent | 20% |
| Oleyl Alcohol (OA) | Bioavailability | 5% |
| | Enhancer | |
| | Bioavailability | 5% |
| Diethylene glycol monoethyl | Enhancer | |
| ether (DGEE) | Total | 100% |

A lipid based composition according to the above formulation was developed using the following procedure.
Make formulation in a 2-L glass beaker.
Weigh and add in GM, PG, OA and DGEE
Mix at moderate rpm for 10 min
Weigh and add 5LDMN. Mix till dissolve.
Stir at moderate rpm till dissolve.
Sit overnight.

### Example 2: Synthesis of 5 Demethyl Nobiletin

FIG. 10 illustrates the synthesis of 5-hydroxy 6,7,8,3′,4′ pentamethoxyflavone (5 demethyl nobiletin) In the two-step synthesis, the starting material starts from PMFs such as nobiletin, because the natural contents of PMFs in citrus peels are much richer than that of hydroxylated PMFs. In the preparation of 5-hydroxylated PMFs (also called 5-demethyl PMFs if there is a methoxy group in the parent PMFs.), one step ether hydrolysis in an acid catalyzed condition such as hydrochloric acid (aqueous HCl solution or dry HCl gas). Other Lewis acids can be used such as BCl3, BF3 for a quicker reaction but usually results low purity and low yield. HCl either gas or concentrated aqueous solution is broadly used because its high selectivity at 5-position and also a relative green process.

5 kg of citrus extract (PMF content >60%) was suspended in 25 kg of 95% ethanol solution, to which concentrated hydrochloric acid (15 kg) was added while heating till the solution turned clear (non-cloudy except solid particles).

The resulting solution/suspension was refluxed for 24-48 hours depending on the conversion of nobiletin or other PMFs to 5-demethylnobiletin or 5-demethylated PMFs monitored by TLC (thin-layer chromatography) or HPLC (high performance liquid chromatography) to illustrate a complete or minimum of 95% conversion. Then the heating was stopped (the heating source was removed if doable) and the solution was cooled to ambient temperature. To the cooled solution/suspension was concentrated in vacuo, and then added deionized water (50 kg) and 50 kg DCM. The organic layer was separated after shaking and layer settling. To the organic layer, 20% of sodium carbonate aqueous solution (w/v) was added to adjust pH to 7. Again, the organic layer was separated after shaking and layer settling, and water was added to the separated DCM solution, to which after separation, brine was added. Eventually, the organic layer was separated, to which anhydrous sodium sulfate was added. Filtration and concentration yielded crude 5OHPMF intermediate, which was subject to normal phase chromatography or (repeated) crystallization depending on the purity score of 5—OHPMFs by HPLC analysis.

### Example 2: Chemical Synthesis of 5 Lauryl 6,7,8,3′,4′ Pentamethoxyflavone

The hydroxyl groups of hydroxylated PMFs can be converted to an ester under the reagents and conditions of chemical esterification, such as acid catalysis and heating, acyl chloride and a base, fatty acid anhydride and a base, or an insitu generated acyl halides from fatty acid and halide phosphates, acetyl halides etc under with or without the presence of a base. The conversion rate of 5-dimethyl nobiletin to its acyl ester can be optimized to reach over 99%.

5—OHPMFs (2 kg) in the first step were dissolved in DCM (50 kg), to which triethylamine (1 kg) was added. The resulting solution was cooled to 0 C. While under nitrogen flow, lauryl chloride (1.8 kg) was then added to the resulting solution at a temperature range of 0-10 C.

The resulting mixture was reacted for 18 hours under nitrogen protection and monitored by TLC. Upon the completion of the acylation, more DCM (25 kg) was added to the reaction mixture. To the yielded reaction mixture, sodium bicarbonate aqueous solution (5%, w/v, 40 kg) was added. The organic layer was washed again with water (40 L) and brine (20 kg) after separation of the organic layer.

Anhydrous sodium sulfate (3 kg) was then added to the separated organic layer, which was then filtered and concentrated in vacuo to dryness yield crude nobiletin lauryl ester. The obtained crude ester was subject to a normal phase column with an eluting system of EtOAc/hexanes or DCM/hexanes to generate high purity of lauryl ester of 5-demethyl nobiletin and its related 5-demethylated analogs (>98% purity).

1. A compound of formula (I), wherein the compound comprises a derivative of a 5-hydroxylated polymethoxyflavone, wherein R5 is a pharmaceutically acceptable lipid carrier selected to improve the oral bioavailability of the 5-hydroxylated polymethoxyflavone and wherein R6, R7 and R4′ are OCH3, wherein R3, R8 and R3′ are OCH3 or H and R2′, R5′ and R6′ are H.

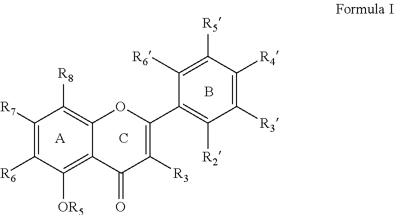

2. The compound according to claim 1, wherein the 5-hydroxylated polymethoxyflavone is selected from 5-hydroxy-6,7,8,3′,4′-pentamethoxyflavone, 5-hydroxy-6,7,8,4′-tetramethoxyflavone, 5-hydroxy-6,7,3′,4′-tetramethoxyflavone and 5-hydroxy 3,6,7,8,3′,4′-hexamethoxyflavone.

3. The compound according to claim 2, wherein the Log P value of the compound is greater than 5 and the solubility in triglycerides is greater than 50 mg/mL.

4. The compound according to claim 3, wherein the oral bioavailability of the derivative of the 5-hydroxylated polymethoxyflavone prodrug is at least 2-fold higher than that of the unmodified 5-hydroxylated polymethoxylated flavone.

5. The compound according to claim 3 wherein the lipid carrier is selected from a long chain fatty acid, a phospholipid or a glyceride.

6. The compound according to claim 5, wherein the lipid carrier is a long chain fatty acid selected from lauric acid, myristic acid, palmitic, myristoleic, palmitoleic, oleic, linoleic and linolenic acid.

7. The compound according to claim 6, wherein the lipid carrier is a glycerophospholipid.

8. The compound according to claim 6 wherein the lipid carrier is a glyceride.

9. A compound of formula (II), wherein the compound comprises a derivative of a 5-hydroxylated polymethoxyflavone, wherein R5 is a pharmaceutically acceptable hydrophilic carrier selected to improve the water solubility of the 5-hydroxylated polymethoxylated flavone and wherein R6, R7 and R4′ are OCH3, wherein R3, R8 and R3′ are OCH3 or H and R2′, R5′ and R6′ are H.

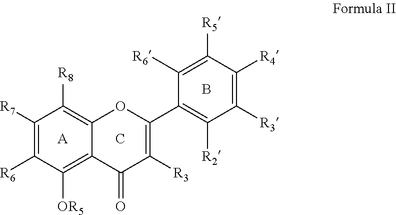

10. The compound of claim 9 wherein the water solubility is greater than 10 mg/mL.

11. The compound according to claim 10, wherein the hydrophilic carrier is a phosphate, glycoside, PEGylate or carbonate functional group.

12. A pharmaceutical lipid-based composition comprising a therapeutically effective amount of the compound of claim 1 and one or more solvents, wherein the one or more solvents promote lymphatic uptake.

13. The composition of claim 12 comprising between 1% and 30% of the 5HPMF derivative by weight and preferably between 5% and 20% by weight.

14. The composition of claim 13, wherein the solvent has an HLB value of less than 5.

15. The composition of claim 14 wherein the one or more solvents is an ester of glycerol, glycol or fatty alcohol and a fatty acid wherein the one or more solvents has an HLB value less than 5.

16. The composition of claim 15 further comprising a bioavailability enhancer.

17. The composition of claim 16 wherein the bioavailability enhancer is oleyl alcohol.

18. The composition of claim 16 wherein the bioavailability enhancer is an ether of diethylene glycol.
